(19) United States
(12) Patent Application Publication
(10) Pub. No.: US 2005/0119866 A1
(43) Pub. Date: Jun. 2, 2005

(54) Medical parameter processing system

(76) Inventor: John R. Zaleski, West Brandywine, PA (US)

Correspondence Address: Alexander J. Burke;Intellectual Property Department, 5th Floor, 170 Wood Avenue South, Iselin, NJ, 08830, US

(21) Appl. No.: 10/970,565

(22) Filed: Oct. 21, 2004

Related U.S. Application Data
(60) Provisional application No. 60/520,016, filed on Nov. 14, 2003.

(51) Int. Cl. H04B 15/00; G06F 15/00; H03F 1/26; G01N 33/50; G01N 33/48; G06F 19/00; G06F 17/60

(52) U.S. Cl. 702/189; 702/19

(57) ABSTRACT

A processing system for patient medical parameters includes a communication interface for acquiring patient parameter data comprising a medically significant signal from a patient monitoring device attached to a patient. A transform processor converts the medically significant signal into a plurality of components using a transform. A filter filters the components to exclude components based on criteria to provide filtered components. An inverse transform processor inverse-transforms the filtered components to provide a representation of the medically significant signal.

This application claims priority based on provisional patent application Ser. No. 60/520,016 which was filed on November 14, 2003.

### FIELD OF THE INVENTION

The present invention relates generally to the field of biomedical data processing, and more specifically to a method of storing and retrieving clinical telemetry data.

### BACKGROUND OF THE INVENTION

As medical equipment becomes more sophisticated and allows seamless connectivity from the point of care to enterprise information management systems, more patient data collected at the point of care becomes available for remote viewing and analysis within the electronic medical record. Such systems may provide at least a subset of the valuable clinical information gathered at the point of care to anywhere within the enterprise, on site and off site, with viewing capability available, even at a clinician's home.

In communicating raw clinical data from departmental to enterprise information systems within a healthcare enterprise, care is required regarding the amount of data that is acquired and saved. Too little data results in possibly significant physiological events not being in the acquired and saved data. Too much data overloads the system in terms of data throughput over the network and the required amount of data storage. In addition, too much data also may impair a physician's ability to effectively analyze such a large amount of data for the possibly relatively rare physiological events embedded within that large amount of data. Ultimately, the goal is to help the patient. Any method that does not aid in clinical diagnosis or treatment may even impede clinical progress, which would be detrimental to care.

In a typical critical care medical facility, a raw clinical data stream, having a substantial amount of data is continuously generated by bedside patient monitors. Telemetry data obtained from the patient monitors is normally not stored in its entirety. Instead, in standard practice, a flow sheet or assessment sheet is maintained in which a clinician, such as an anesthesiologist, nurse, respiratory therapist, etc., enters, at regular time intervals, important patient status information, e.g. including the heart rate, respiratory rate, and many other key parameters. Thus, these parameters are monitored continuously but recorded at discrete time intervals in the clinical record. In the least sophisticated systems, this information is recorded by hand using a paper flow sheet or assessment sheet at the patient's bedside. The assessment sheet remains with the patient throughout the stay at the hospital. Once completed, the paper record becomes a part of the patient's permanent record.

As information technology progresses, much of the paper based record keeping system is being supplanted by an electronic record, in which clinicians record information from patient monitoring systems, either manually or automatically, in electronic form. The data in this electronic form of assessment sheet is maintained in a central location in the hospital. The electronic assessment sheet remains associated with the patient throughout the phases of diagnosis and treatment in the hospital. The types of information which can be recorded, transmitted, and viewed within a typical health information system include administrative and demographic information as well as clinical results and telemetry data as would be entered in a paper assessment sheet.

A benefit of an electronic system is that, unlike the paper record, the electronic medical record may be accessed from many different locations without physically retrieving the patient's hardcopy information from a particular department. Loss of information is unlikely, and the use of electronic medical records establishes a standard approach for recording patient information. Each department conforms to specific standards in terms of the types and quality of information being recorded for each patient. Browser based technology derived from world wide web applications further simplifies medical record inspection, in terms of both viewing convenience and reducing the delays associated with retrieving the paper record. The net result is that clinicians can readily obtain patient information when required and where required. In addition, two way communication between the health information system and clinical systems enables the relatively error free retrieval of other patient information such as their medical record number and insurance information without adding further delay or introducing errors into the patient's record within the departmental system.

Bedside monitors typically can generate detailed information at intervals down to fractions of a second. In typical systems, a portion of the generated data is recorded, i.e. most of this information is discarded. More specifically, this data is recorded at predetermined time intervals (e.g. 15 minute intervals), in a manner similar to paper assessment sheets. An inherent compromise exists in terms of the size of the interval and the capturing of relatively important data from the bedside monitors. If the recording interval is too large, events of relatively short duration but high importance such as heart rate spikes or respiratory rate increases may be missed and not recorded within the electronic medical record. On the other hand, if the recording interval is too small the throughput of data across the hospital computing network and the storage requirements in a mass storage device of the medical record increase. The medical record becomes cumbersome and filled with much useless information, possibly even rendering the system unusable.

For example, during the course of a single hour, the total quantity of individually unique results can easily exceed five thousand values. Over the course of an eight hour shift, this can grow to nearly forty thousand values for a single patient and single physiological parameter. Multiplying these numbers by thirty or more patients and more than one physiological parameter per patient makes clear that the data quantity problem can become unmanageable. Automatic data compression is used to assist in the storage of such data within an associated long term archive. However, with the integration of clinical systems and a health information system via a long term clinical record, it is necessary to strike a balance between a system wide data deficit and a system wide data surplus.

Even in an inefficient health information system, the intention of the administrators and users is not to store a complete record of the patient telemetry data. One of the clinician's roles is to identity unimportant information and to record information which is deemed important for the clinical record of that patient. This goal, however, is impractical in a real world clinical environment. Clinicians are frequently moving from patient to patient with their primary focus being on patient care and not on dedicating their time and energy to full time data identification and collection. Clinical information tools available today do provide for filtering of repeated information, reducing the chance that redundant results are continually sent to the long term clinical record. Unfortunately, data that does not repeat or otherwise have non-repeating variations are not accommodated by this filtering approach. Filtering approaches use a simple comparison with a previous data value to determine whether the newer value should be excluded from transmission to the long term record. The simple filtering method treats data that is noisy or rarely repeats as ‘important’ and in such a case unimportant data points are transmitted to the long term record.

Results stored in the assessment sheet for the purpose of clinical reporting are sometimes inadequate as well, as can be appreciated by reference to FIG. 1. The data depicted in FIG. 1 is derived from an individual via a bedside monitor which measures patient heart rate at predetermined time intervals. This monitor produces a result that is transmitted to the long term clinical record. Curve 1 is a plot of raw heart rate data taken from an individual at fifteen second intervals, and is displayed over the course of approximately a seventy minute period, i.e. approximately 280 data points.

Overlaid on the curve 1 are data points 2, 8, 9, 10 and 11 of the assessment sheet curve 12. These data points reflect those values that normally are recorded using a fifteen minute update time interval of the clinical assessment sheet, as would be typical in a surgical intensive care environment. Curve 12 is a plot through the data points 2, 8, 9, 10 and 11. As seen in FIG. 1, the five data points 2, 8, 9, 10 and 11 in the assessment sheet recordings omit a large amount of patient telemetry data. These recordings provide no indication as to the typical value of heart rate, its extent during the course of the entire recording interval, or episodes indicative of exceeding the range of expected values. For example, the raw data collected at fifteen second intervals show that at point 3 a minimum pulse rate is recorded as 55 beats per minute at approximately minute 39 during the recording trace. At point 4 a maximum pulse of 105 beats per minute was measured at approximately minute 52. The two extreme values at points 3 and 4 are absent from the standard assessment sheet recording 2.

A typical data management approach to the foregoing observations is that by maintaining a running record of the minimum, the maximum, and the mean signal value, the necessary information can be provided. However, this additional signal value information, while providing more insight into the range of the values over the course of the measurement period, does not provide insight into the behavior and trend of the raw signal data over time. The average value 5, together with the signal minimum 3 and maximum signal 4, add an additional three data points to the assessment sheet recording. If the exact time 6 of the minimum rate 3 and the exact time 7 of the maximum rate 4 are added, an additional two data points become part of the record for this patient. However, variations in signal behavior, and short term responses to stimuli such as drug interactions are still missing from the assessment sheet, even with this additional information.

The depiction of raw results shown in the curve 1 is representative of approximately 280 data points. In order for the collected telemetry data to be of value, a system is needed that may utilize this data without requiring an automated system to store, and an end user to view, the complete record of the raw information that has been collected for an individual patient, while still providing some insight into the character and trend of the original signal data.

### BRIEF SUMMARY OF THE INVENTION

In accordance with principles of the present invention, a processing system for patient medical parameters includes a communication interface for acquiring patient parameter data comprising a medically significant signal from a patient monitoring device attached to a patient. A transform processor converts the medically significant signal into a plurality of components using a transform. A filter filters the components to exclude components based on criteria to provide filtered components. An inverse transform processor inverse-transforms the filtered components to provide a representation of the medically significant signal.

### BRIEF DESCRIPTION OF THE DRAWING

In the drawing:

FIG. 1 (prior art) is a graph depicting the relationship between raw telemetry data and selected summary data as recorded in a typical long term medical record context;

FIG. 2 illustrates the process of computing discrete wavelet transforms using the Haar basis function;

FIGS. 3A, 3B, 3C and 3D, examined in sequence, illustrate the discrete wavelet transform process of computing the averages and differences for each of the supplied signal components for a signal vector containing eight data elements;

FIGS. 3E and 3F, examined in sequence, illustrate the computation of the averages and differences of those averages and differences computed for the eight data elements as illustrated in FIGS. 3A through 3D;

FIG. 3G illustrates the computation of the average and difference for those averages and differences previously calculated and illustrated in FIGS. 3E and 3F;

FIG. 3H illustrates the discrete wavelet transform vector for the supplied signal of FIGS. 3A through 3D;

FIG. 4 is a comparative view of wavelet coefficients needed to provide complete signal and threshold levels according to the principles of the present invention;

FIG. 5 is a comparative view of wavelet coefficients needed to provide revised signal and threshold levels according to the principles of the present invention;

FIG. 6 illustrates the set of Haar wavelet coefficients generated from the data set depicted in FIG. 1;

FIG. 7 depicts a reconstruction of the signal illustrated in FIG. 1 using a first set of discrete wavelet coefficients according to the principles of the present invention;

FIG. 8 depicts a reconstruction of the signal illustrated in FIG. 1 using a second set of discrete wavelet coefficients according to the principles of the present invention;

FIG. 9 depicts the temporal profile of forced inspired oxygen fraction, FiO₂, for a typical patient;

FIG. 10 depicts the FiO₂ wavelet coefficients created according to the principles of the present invention for the data depicted in FIG. 9;

FIG. 11 is a reconstruction of the signal depicted in FIG. 9 using a set of discrete wavelet coefficients according to the principles of the present invention;

FIG. 12 depicts the mandatory respiratory rate setting versus time for a typical patient;

FIG. 13 depicts the wavelet coefficients created according to the principles of the present invention for the data depicted in FIG. 12;

FIG. 14 is a reconstruction of the signal depicted in FIG. 12 using a set of discrete wavelet coefficients according to the principles of the present invention;

FIG. 15 depicts the respiratory rate in breaths per minute versus time for a typical patient;

FIG. 16 is a depiction of reconstructed signals derived from the data of FIG. 15, based on various wavelet exclusion threshold values according to the principles of the present invention;

FIG. 17 is a graph depicting a complete set of wavelet coefficients for the data depicted in FIG. 15, created according to the principles of the present invention;

FIG. 18 depicts the wavelet coefficients remaining after application of a twenty percent exclusion threshold to the data depicted in FIG. 17;

FIG. 19 is a block diagram depicting a system for the storage of data and the computation of discrete wavelet transform coefficients according to the principles of the present invention;

FIG. 20 is a block diagram of a system depicting the transfer of assessment sheet results and discrete wavelet transform coefficients to the long term clinical record according to the principles of the present invention; and

FIG. 21 is a graphical user interface utilized by a discrete wavelet transform processor constructed according to the principles of the present invention.

### DETAILED DESCRIPTION OF THE INVENTION

The present invention utilizes a discrete wavelet transform to provide a system for reconstructing or approximating a data signal or function. The disclosed wavelet transform provides time and magnitude localization of data signal specifics. More specifically, a data signal is partitioned into successive blocks containing a predetermined number of signal samples. Within each block, the data signal is encoded into coefficients representing magnitude details at differing time resolutions. This provides advantages (to be described below) when reconstructing time varying, nonstationary processes typically occurring in biomedical telemetry data. In the illustrated embodiment, the discrete wavelet transform calculation is conducted with respect to a Haar basis function, in which individual averages and differences, or details, are computed with respect to the raw signal data. One skilled in the art understands that other basis functions may also be used to perform the wavelet transformation, understands the advantages and limitations of the different basis functions, and understands how to select an appropriate basis function for a particular application.

For example, a small sample signal vector f^{T} of raw data collected from a patient may take the form set forth in Equation 1:
f^{T}=[5 −2 3 ] [1]

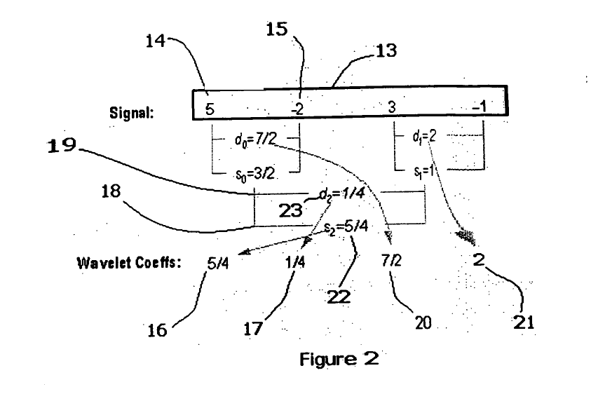

The process of computing wavelet coefficients from the vector f^{T} described by Equation 1 is understood by referring to FIG. 2. The signal 13 is decomposed into a series of averages and differences, where the average sᵢ is computed according to normal convention, and the difference dᵢ is computed as half the arithmetic difference between any two adjacent raw signal values. Thus,

$$\begin{matrix}{s}_{i} = \frac{{f}_{i} + {f}_{i + 1}}{2} , and & [ 2 ] \\ {d}_{i} = \frac{{f}_{i} - {f}_{i + 1}}{2} . & [ 3 ]\end{matrix}$$

The computation illustrated in FIG. 2 calculates averages sᵢ of each raw sample and its immediate neighbor. For example, samples 14 and 15 are averaged according to equation [2]. In the case, the average s₀ of samples 14 and 15 is 3/2. Similarly, differences dᵢ are calculated between each raw sample and its immediate neighbor. Differences dᵢ between neighboring raw samples are computed according to equation [3], as the arithmetic difference between the two samples divided by two. In the case of samples 14 and 15, for example, this computation yields a difference d₀ of 7/2. These calculations are repeated for each pair of raw samples. Once each values of sᵢ and dᵢ are computed for each pair of raw samples, the same calculations, i.e. average s and difference d, of neighboring values of the average s are computed until an ensemble average value S₂ 22 (representing the average of the entire sample set of the signal 13) and difference value d₂ 23 is determined. The first wavelet coefficient 16 is given by the ensemble average S₂, covering the entire signal interval i.e. the most coarse level 18. The remaining coefficients represent differences, or details, which when combined with the ensemble average S₂ may be used to recreate the original signal vector f^{T}. For example, the next wavelet coefficient 17 corresponds to the difference value d₂ of the averages at the preceding or finer scale 19. In general, the remaining coefficients, for example coefficients d₀20 and d₁21, follow the pattern of the differences between the averages at finer and finer scale or resolution. Thus, the vector b^{T} of wavelet coefficients for the data sample 13 is:

$${b}^{T} = [ \begin{matrix}\frac{5}{4} & \frac{1}{4} & \frac{7}{2} & 2\end{matrix} ] \qquad [ 4 ]$$

The relationship between the wavelet coefficients and the raw signal data may be expressed in matrix form:
f=H₄b [5]
where H₄ represents a 4×4 Haar matrix having the form:

$${H}_{4} = [ \begin{matrix}1 & 1 & 1 & 0 \\ 1 & 1 & - 1 & 0 \\ 1 & - 1 & 0 & 1 \\ 1 & - 1 & 0 & - 1\end{matrix} ] \qquad [ 6 ]$$

Given available raw signal data f, the wavelet coefficients b may be found directly as follows:
b=H₄⁻¹ [7]
The Haar matrix H₄ may also be inverted using standard methods.

The creation of the Haar matrix follows a predictable pattern as the number of rows and columns increases. However, by applying the Haar transform, the size of the matrix increases exponentially according to a scale of p=2n, where n is a positive integer. Thus, in the Haar basis function, the quantity of data conforms to this scale as well. The 8×8 Haar basis function H₈ basis is:

$${H}_{8} = [ \begin{matrix}1 & 1 & 1 & 0 & 1 & 0 & 0 & 0 \\ 1 & 1 & 1 & 0 & - 1 & 0 & 0 & 0 \\ 1 & 1 & - 1 & 0 & 0 & 1 & 0 & 0 \\ 1 & 1 & - 1 & 0 & 0 & - 1 & 0 & 0 \\ 1 & - 1 & 0 & 1 & 0 & 0 & 1 & 0 \\ 1 & - 1 & 0 & 1 & 0 & 0 & - 1 & 0 \\ 1 & - 1 & 0 & - 1 & 0 & 0 & 0 & 1 \\ 1 & - 1 & 0 & - 1 & 0 & 0 & 0 & - 1\end{matrix} ] \qquad [ 8 ]$$

The number of rows and columns contained within a Haar Hₚ basis follows in accord with the value of 2n. The H₈ basis matrix transforms sample vectors f having 8 samples to transform vectors b having 8 coefficients.

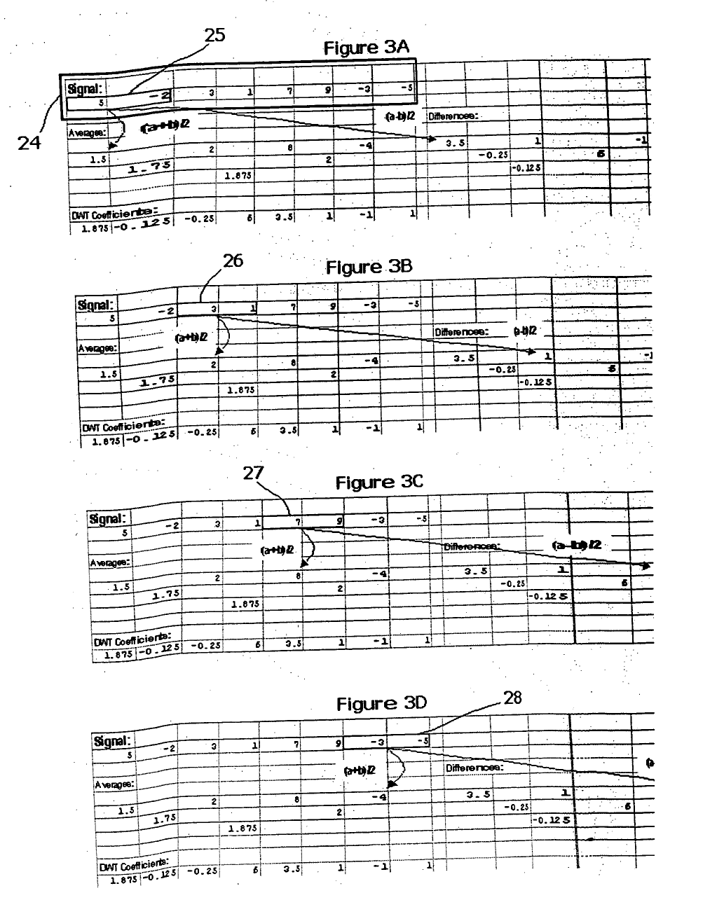

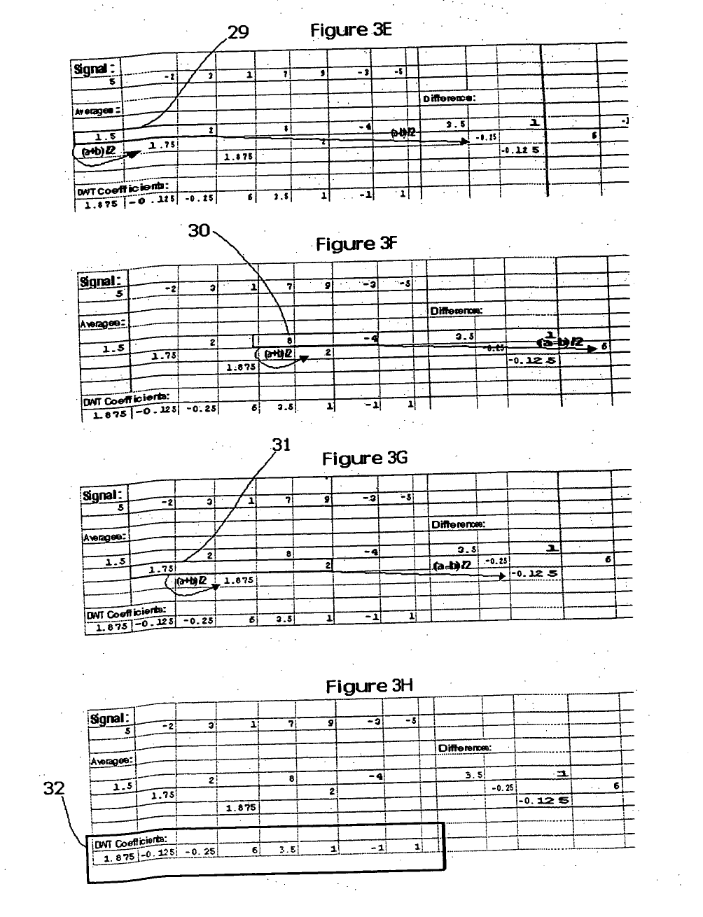

Referring also to FIGS. 3A through 3H, another example of discrete wavelet transform creation is set forth. The raw signal 24 includes a signal vector f^{T} of eight elements as defined by the equation:
f^{T}=[5 −2 3 1 7 9 −3 −5] [9]

As seen in FIGS. 3A through 3D the discrete wavelet transform calculation begins by computing the averages sᵢ according to equation [2] and differences dᵢ according to equation [3] for each of the signal 24 components. For the signal 24, there are four sets 25, 26, 27 and 28 of neighboring values to which equations [2] and [3] are applied, resulting in four average values of s₀, S₁, S₂ and S₃, as well as four difference values of d₀, d₁, d₂ and d₃. In this example, the value of s₀ is 1.5; the value of s₁ is 2; the value of S₂ is 8 and the value of S₃ is −4. Similarly, the value of d₀ is 3.5; the value of d₁ is 1; the value of d₂ is −1 and the value of d₃ is 1.

FIGS. 3E and 3F illustrates how equations [2] and [3] are applied to the average values s₀, S₁, S₂, S₃ calculated in FIGS. 3A through 3D. Thus, the value of so and the value of s₁ are used in cell 29 to create the average value of s₄ (equation [2]), and the difference value of d₄ (equation [3]). The value of S₂ and the value of S₃ are used in cell 30 to create the average value of S₅, and the difference value of d₅. In this example, the value of S₄ is 1.75 and the value of s₅ is 2, while the value of d₄ is −0.25 and the value of d₅ is 6.

As best seen in FIG. 3G, the average values of S₄ and S₅ are used in cell 31 and again equations [2] and [3] are applied to the values S₄ and S₅ to create an ensemble average value S₆ of 1.875 and a difference value d₆ of −0.125. The resulting discrete wavelet transform vector created for raw signal 24 appears in cell 32 and consists of the ensemble average S₆ and the difference or detail values from most coarse to finest: i.e. d₆, d₅, d₄, d₃, d₂, d₁, and d₀. Thus, the vector b^{T} of wavelet coefficients associated with the signal 24, generated using the H₈ basis, is:
b^{T}=[1.875 −0.125 −0.25 6 3.5 1 −1 1] [10]

As may be seen from equations [5] and [7] above, given a complete set of wavelet coefficients b, a signal f may be reconstructed with no loss. One feature of the wavelet coefficients is that they establish the relative scale of the absolute differences with respect to the overall raw signal average. That is, the difference terms dᵢ provide an indication of a deviation of a signal from the ensemble average value sᵢ at respective levels of coarseness or fineness. In terms of reproducing the raw signal 24, the values of the wavelet coefficients establish their relative impact on the overall signal: the smaller the coefficient, the lower the impact on the overall signal. Thus, compression of the original signal may be achieved, with some degree of loss, by discarding a subset of these coefficients based on the establishment of a sensitivity threshold (described in more detail below).

Discarding potentially important information from the raw signal can be detrimental and provides a clinician with incomplete patient data. However, wavelet transformation provides the capability to automatically record the complete data signal while filtering out relatively unimportant details. This ensures that communication of the important data elements between the patient monitoring and treatment devices in the clinical environment and the storage devices in the health enterprise does not overwhelm the system. Further, any amount of the data, from a complete ensemble to that representing detailed temporal changes, may be inspected as desired by clinicians and researchers without requiring that the complete record of data be retrieved from the data repository in a single request.

The magnitude of the wavelet coefficients provides insight into the level of contribution they make to the character of the overall raw signal 24. Hence, by omitting certain coefficients, or substituting zero coefficients for them, it becomes possible to exclude noise, artifact, or other components that are judged to be of minor influence to the overall raw data sample. As an example of this capability, Table 1 presents an array of representative wavelet coefficients. The first column is the independent variable: time. Subsequent sets of columns define corresponding sets of Haar basis wavelet coefficients and the resulting signal values. The second set of columns represent wavelet coefficients to which no threshold is applied; the third set of columns represent wavelet coefficients to which a 10% threshold is applied; the fourth set of columns represent wavelet coefficients to which a 20% threshold is applied; and the fifth set of columns represent wavelet coefficients to which a 30% threshold is applied.

The absolute threshold values are computed by multiplying the selected threshold percentage by the largest wavelet coefficient present. For example, a 10% threshold is calculated by multiplying the largest coefficient magnitude value of −4 (occurring at time 8.0) by 0.1 (i.e. 10%) to obtain an absolute threshold value of 0.4. That is, the constraint imposed by the 10% threshold is that the absolute value of allowed wavelet coefficients be greater than 0.4. For the case of a 10% threshold, one wavelet coefficient (−0.250 occurring at time 3.0) is discarded. The discarded coefficient is set to zero so that its contribution is ignored for the purposes of signal reconstruction. The resulting wavelet coefficients are listed in the first column in the ‘10% Threshold’ set of columns. A signal, reconstructed from these wavelet coefficients, is listed in the second column in the ‘10% Threshold’ set of columns, and the error between the reconstructed signal and the actual signal (from the second column in the ‘No Threshold’ set of columns) is listed in the third column in the ‘10% Threshold’ set of

TABLE 1
Wavelet coefficients for complete signal and threshold levels

| | No Threshold | | 10% Threshold | | | 20% Threshold | | | 30% Threshold | | |
|---|---|---|---|---|---|---|---|---|---|---|---|
| | Wavelet | | Wavelet | | | Wavelet | | | Wavelet | | |
| time | Coefficients | Signal | Coefficients | Signal | \|Error\| | Coefficients | Signal | \|Error\| | Coefficients | Signal | \|Error\| |
| 1.0 | 3.125 | 5.000 | 3.125 | 5.250 | 0.250 | 3.125 | 5.250 | 0.250 | 3.125 | 5.250 | 0.250 |
| 2.0 | −1.375 | −2.000 | −1.375 | −1.750 | 0.250 | −1.375 | −1.750 | 0.250 | −1.375 | −1.750 | 0.250 |
| 3.0 | −0.250 | 3.000 | 0.000 | 2.750 | 0.250 | 0.000 | 2.750 | 0.250 | 0.000 | 1.750 | 1.250 |
| 4.0 | 3.500 | 1.000 | 3.500 | 0.750 | 0.250 | 3.500 | 0.750 | 0.250 | 3.500 | 1.750 | 0.750 |
| 5.0 | 3.500 | 7.000 | 3.500 | 7.000 | 0.000 | 3.500 | 7.000 | 0.000 | 3.500 | 8.000 | 1.000 |
| 6.0 | 1.000 | 9.000 | 1.000 | 9.000 | 0.000 | 1.000 | 9.000 | 0.000 | 0.000 | 8.000 | 1.000 |
| 7.0 | −1.000 | −3.000 | −1.000 | −3.000 | 0.000 | −1.000 | −3.000 | 0.000 | 0.000 | −3.000 | 0.000 |
| 8.0 | −4.000 | 5.000 | −4.000 | 5.000 | 0.000 | −4.000 | 5.000 | 0.000 | −4.000 | 5.000 | 0.000 |

At the 20% Threshold level, the absolute threshold value is 0.2 multiplied by −4, yielding an absolute threshold of 0.8. In this case, too, the coefficient occurring at time 3.0 is discarded, i.e. is set to zero. Thus, the reconstructed signal and errors for the 20% Threshold level are the same as for the 10% Threshold level. The differences between the complete ensemble of wavelet coefficients and the threshold filtered wavelet coefficients result in a maximum error or deviation of 0.250 between the reconstructed signal and the original signal.

At the 30% threshold level, the absolute threshold value is 0.3 multiplied by −4, yielding an absolute threshold of 1.2. In this case, three coefficients (values −0.250, 1.000 and −1.000 at times 3.0, 6.0 and 7.0 respectively) are discarded, i.e. set to zero. The filtered wavelet coefficients are listed in the first column in the ‘30% Threshold’ set of columns. A signal reconstructed from the filtered wavelet coefficients is listed in the second column of the ‘30% Threshold’ set of columns, and the difference between the reconstructed signal and the actual signal is listed in the third column in the ‘30% Threshold’ set of columns. In this case, the error or deviation between the original signal and the reconstructed signal is no greater than 1.25.

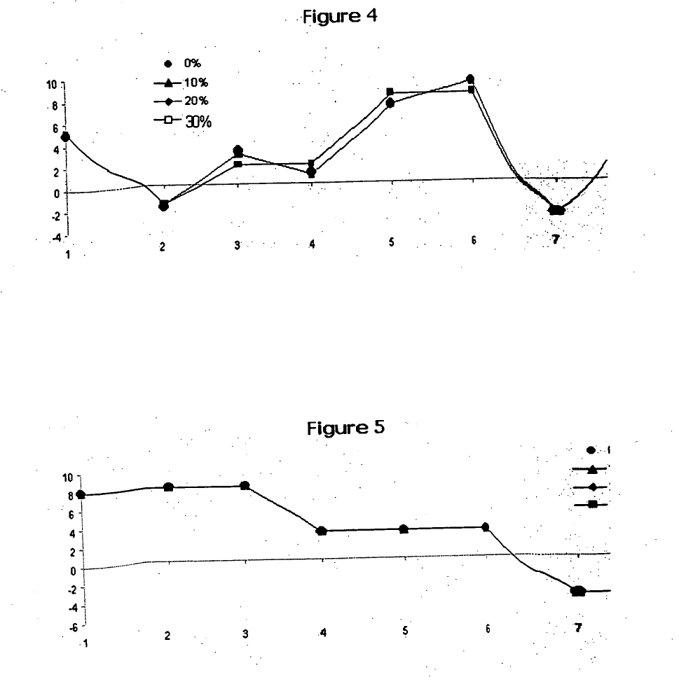

The general impact of discarding coefficients from the wavelet coefficient vector is to reproduce a signal which is an approximation of the original signal. The higher the threshold, the higher the error between the reconstructed signal and the actual signal. Conversely, as the threshold for discarding coefficients approaches zero, the difference between the reconstructed signal and the original signal approaches zero. FIG. 4 provides a comparative view of the data appearing in Table 1, displaying the aforementioned reconstructed signals on a single graph. In FIG. 4 the signal data points for the four reconstructed signals appear to be close to or overlaying one another.

Depending on the shape, repetitiveness, noise content and other behavior of the original signal, the degree of loss that results from discarding wavelet coefficients may 10 or may not be acceptable to the end user. However, for most telemetry applications, there is not a significant difference between the lossy, high threshold, cases and the lossless, no threshold, case.

In the case of a predictable or repetitive signal, the discarding of wavelet coefficients can have a trivial effect on the reconstruction of the original signal. This 15 latter case can be illustrated effectively with the aid of a different type of raw signal data characteristic. The data is presented in Table 2, and plotted in FIG. 5.

TABLE 2
Wavelet coefficients for revised signal and threshold levels

| | No Threshold | | 10% Threshold | | | 20% Threshold | | | 30% Threshold | | |
|---|---|---|---|---|---|---|---|---|---|---|---|
| | Wavelet | | Wavelet | | | Wavelet | | | Wavelet | | |
| time | Coefficients | Signal | Coefficients | Signal | \|Error\| | Coefficients | Signal | \|Error\| | Coefficients | Signal | \|Error\| |
| 1.0 | 3.125 | 8.000 | 3.125 | 8.000 | 0.000 | 3.125 | 8.000 | 0.000 | 3.125 | 8.000 | 0.000 |
| 2.0 | 3.625 | 8.000 | 3.625 | 8.000 | 0.000 | 3.625 | 8.000 | 0.000 | 3.625 | 8.000 | 0.000 |
| 3.0 | 1.250 | 8.000 | 1.250 | 8.000 | 0.000 | 1.250 | 8.000 | 0.000 | 1.250 | 8.000 | 0.000 |
| 4.0 | 3.500 | 3.000 | 3.500 | 3.000 | 0.000 | 3.500 | 3.000 | 0.000 | 3.500 | 3.000 | 0.000 |
| 5.0 | 0.000 | 3.000 | 0.000 | 3.000 | 0.000 | 0.000 | 3.000 | 0.000 | 0.000 | 3.000 | 0.000 |
| 6.0 | 2.500 | 3.000 | 2.500 | 3.000 | 0.000 | 2.500 | 3.000 | 0.000 | 2.500 | 3.000 | 0.000 |
| 7.0 | 0.000 | −4.000 | 0.000 | −4.000 | 0.000 | 0.000 | −4.000 | 0.000 | 0.000 | −4.000 | 0.000 |
| 8.0 | 0.000 | −4.000 | 0.000 | −4.000 | 0.000 | 0.000 | −4.000 | 0.000 | 0.000 | −4.000 | 0.000 |

The signal presented in Table 2 and illustrated in FIG. 5 follows a series of three steps, the first having a value of eight for an interval of three time units, the second having a value of three for an interval of three time units, and the third having a value of negative four for an interval of two time units. The full ensemble of wavelet coefficients representing that signal, shown in the second column of Table 2, shows that three coefficient values, at times of 5.0, 7.0 and 8.0, are zero. Hence, these three wavelet coefficients are already treated as discarded, regardless of the threshold level selected. Table 2 shows that no other wavelet coefficients are discarded for the three threshold levels: 10%, 20% and 30%. Thus, even for a 30% threshold level, the original signal is reproduced without loss of critical information. Because no other coefficients are discarded at the three threshold levels, the reconstructed data signals shown in FIG. 5 overlay the raw signal data. The number of wavelet coefficients required to reconstruct this signal with no loss, therefore, is three fewer than the total number of data points contained within the raw signal, or total number of wavelet coefficients which are generated from that signal.

This characteristic is another benefit of the discrete wavelet transform, namely, that the wavelet transformation process itself automatically produces detail coefficients which are zero in locations where no detail exists in the original signal, for example, during periods when the signal has a constant value. Stated differently, the discrete wavelet transform provides a means for representing the original signal with fewer coefficients.

In a clinical context, one measure of the degree of acceptability of a filtered wavelet transformed signal is how accurately the reconstructed signal represents the assessment sheet data. In a typical acute care environment, the assessment data would be the results retained within the long term medical record. Therefore, if the automated signal sampling and data storage reduction features of wavelet transforms can at least convey the assessment sheet values, then the present automated system is providing no less data to the long term record than is already available. However, any additional signal information that is provided can be regarded as beneficial for describing the overall characteristics of the raw signal data. As described above, to provide lossless reproduction, the total number of wavelet coefficients is equal to the total number of data points contained within the original signal. The benefit afforded by the wavelet transform is that it provides a means of determining the relative contribution of each raw signal data point. By excluding certain of these coefficients the original signal may be satisfactorily approximated.

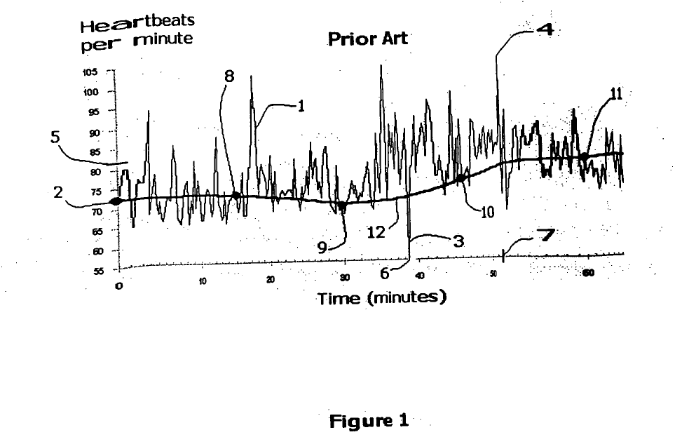

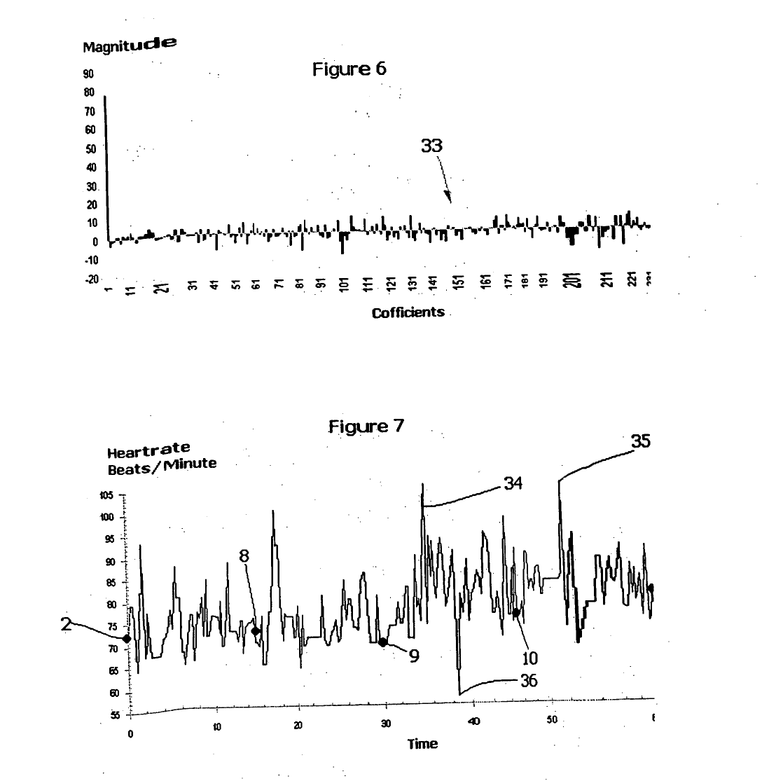

Referring to FIG. 6, a set of Haar wavelet coefficients 33 generated from the raw data set of FIG. 1 is illustrated. An absolute value threshold bₜₕᵣₑₛₕ is specified below which wavelet coefficients are omitted from signal reconstruction. The absolute value threshold is defined mathematically as:
bₜₕᵣₑₛₕ=K×bᵢ^{max} [11]
where bᵢ^{max} is the largest wavelet coefficient within the total set of coefficients under consideration, and K is the percentage level specified in fractional form (for instance: 4% implies K=0.04). When K=0, the complete set the wavelet coefficients are included in the signal reconstruction calculation.

FIG. 7 shows the reconstructed signal 34 resulting from the relatively small threshold K of two percent. As may be seen, the reconstructed signal 34 in FIG. 7 includes less detail than the original signal 1 of FIG. 1. The original assessment sheet data points are present as data points 2, 8, 9, 10 and 11. By setting the threshold level K at two percent, one hundred eighteen of the original two hundred fifty five wavelet coefficients are excluded from the wavelet basis. The set of filtered wavelet coefficients representing the original signal 1 contains one hundred thirty seven wavelet coefficients, or fifty four percent of the total coefficients possible. The approximated signal 34 is substantially coincident with the overlaid assessment sheet data at time points 2, 8, 9, 10 and 11.

The effect of this compression of signal 1 is a relatively small loss in the accuracy of the data that is normally stored in the long term clinical record. However, some insight has been gained into the character of the original signal 1. For example, the maximum signal value 35 appealing in the reconstructed signal 34 is determined to be one hundred six beats per minute, which occurs at approximately fifty two minutes. The minimum signal value 36 appearing in the reconstructed signal 34 is determined to be fifty seven beats per minute, occurring at approximately thirty nine minutes. Contrasting these values with the maximum data point 4 and the minimum data point 3 obtained from the raw data 1 illustrated in FIG. 1 shows close agreement.

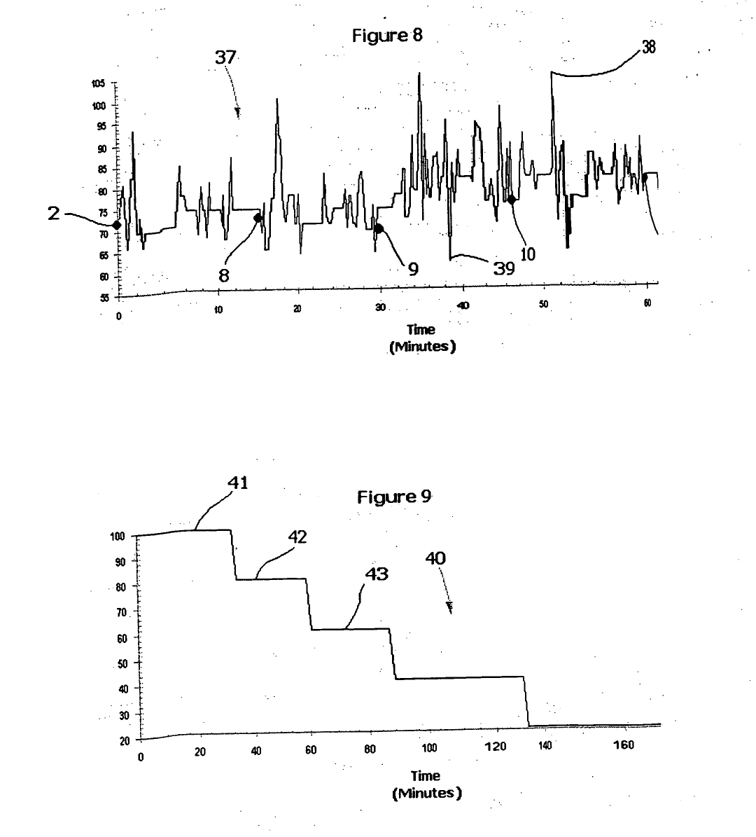

FIG. 8 illustrates a reconstructed signal 37 representing the original signal 1 of FIG. 1 when the threshold K is raised to four percent. As may be seen, the reconstructed signal 37 in FIG. 8 includes less detail than the reconstructed signal 34 in FIG. 7 or the original signal 1 of FIG. 1. The use of a four percent threshold K causes one hundred eighty wavelet coefficients to be discarded in order to approximate the reconstructed signal 37, thus using seventy five wavelet coefficients, or thirty percent of the originally available data points. The original assessment sheet data points 2, 8, 9, 10 and 11 are substantially coincident with the trace of the reconstructed signal 37. The maximum value 38 produced by the reconstructed signal 37 is one hundred five beats per minute occurring at approximately fifty two minutes. The minimum value 39 occurs at thirty-nine minutes and has a value of sixty-one beats per minute. The accuracy of the minimum value 39 in the reproduced signal 37 is off by about five beats per minute, while the maximum heart rate value 38 has remained essentially the same.

The approximation process can be performed with different values for the threshold K resulting in varying degrees of accuracy in the reconstructed signals. Table 3 summarizes the error, or absolute value of the difference, between the recorded assessment sheet value and the value of the reconstructed signal at corresponding times as a function of the threshold K. As the threshold K increases, the error increases. This characteristic is illustrated in the calculation of the root-sum-squared (RSS) error appearing at the bottom of each column. The RSS error is a measure of the ensemble effect of the errors which occur for each time specified within Table 3 for the threshold value K.

### Error Between Recording in Assessment Sheet and the Reconstructed Signal for the Specified Threshold

TABLE 3
Error between recorded assessment sheet and reconstructed signal values
based on specified threshold

| Time | | | |
|---|---|---|---|
| (minutes) | 2% | 4% | 8% |
| 0.00 | 0.73 | 2.48 | 5.39 |
| 14.92 | 2.57 | 1.84 | 5.39 |
| 29.82 | 0.46 | 4.84 | 8.39 |
| 44.98 | 0.20 | 1.55 | 5.11 |
| 59.90 | 0.02 | 0.95 | 2.61 |
| RSS Error: | 2.72 | 6.02 | 12.71 |

Another feature of the present invention is the ability to automatically filter repeated values or results. For nonstationary data, the application of the threshold approach using discrete wavelet transforms makes possible a reduction in artifacts appearing in the raw signal, thereby beneficially reducing the overall quantity of data that is transferred and stored in the long term archive. Data that is repeating can be automatically filtered without applying a heuristic approach such as the comparison of new values with previous values.

The data shown in FIGS. 9 through 11 illustrates the automatic filtering feature of wavelet transformed signals. FIG. 9 shows the temporal profile 40 of forced inspired oxygen fraction (FiO₂) for a specific patient. During the process of weaning a patient from mechanical ventilation, for example, respiratory therapists engage in the process of reducing various kinds of support to a patient in direct proportion to the patient's ability to sustain spontaneous breathing. One specific parameter, FiO₂, is the fraction of oxygen contained in the gas provided to the patient for an inspired breath. The FiO₂ parameter ranges from twenty one to one hundred percent, with most patients starting at or near one hundred percent pure oxygen and eventually being weaned down to an ambient room air oxygen concentration, which is typically on the order of twenty one percent.

Oxygen regulation is a manually controlled process, thus, the reduction in oxygen support is typically done in steps or stages, during which time patients are assessed based on their ability to maintain proper blood oxygenation levels SpO₂ (typically in excess of ninety five percent). The temporal profile of the FiO₂ parameter is typically set in a series of step functions 41, 42 and 43, etc., in which levels are reduced over time. Normally, this parameter is updated in the assessment sheet at the time of each change. The bedside monitor provides an updated value, albeit a constant one, throughout the course of weaning.

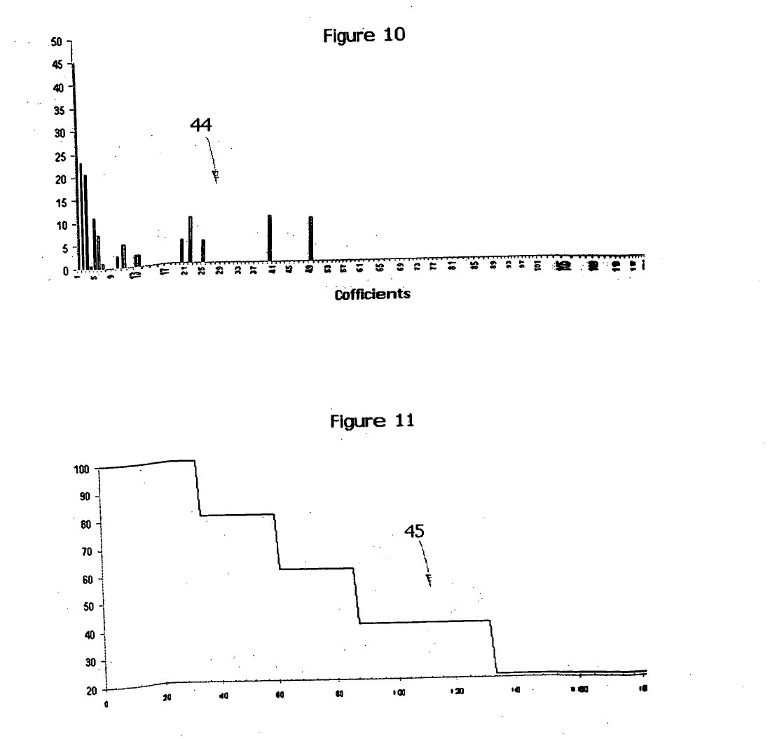

The wavelet coefficients 44 depicted in FIG. 10 are generated using two hundred twenty six signal data points. Some of these coefficients 44 have values which are not necessary to recreate the actual staircase shape of the curve 40 of FIG. 9. FIG. 10 shows the wavelet coefficients 44 corresponding to the waveform 40. Most of the wavelet coefficients have been omitted from the plot 44 because the coefficients having a non-zero value are within the first fifty values. More specifically, fifteen wavelet coefficients have a non-zero value.

Therefore, the original signal 40, represented by two hundred twenty six data points, can be recreated without loss using fifteen wavelet coefficients, as illustrated in FIG. 11, in which the curve 45 is a recreation of the original signal 40 using the fifteen non-zero wavelet coefficients 44. The same method Which was used for the highly time varying heart rate data signal illustrated in FIGS. 6 through 8 has thus been successfully applied to the more predictable FiO₂ data signal without the need to apply any additional heuristic filter to deal with repeating data content. In order to recreate the signal 40 associated with the original two hundred twenty six data points, the typical network need store fifteen data points within the long term record for this particular patient.

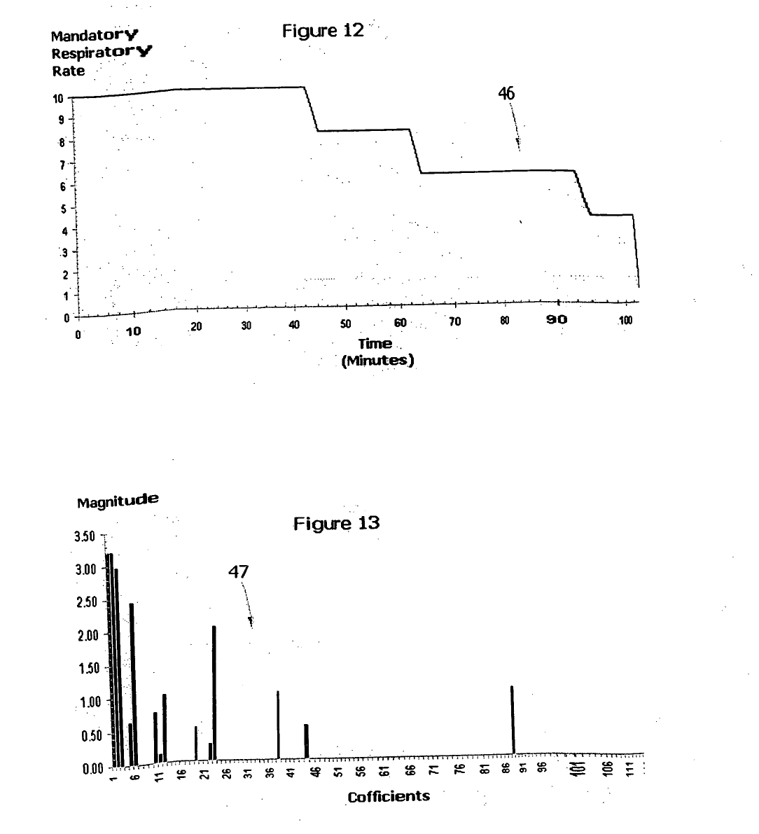

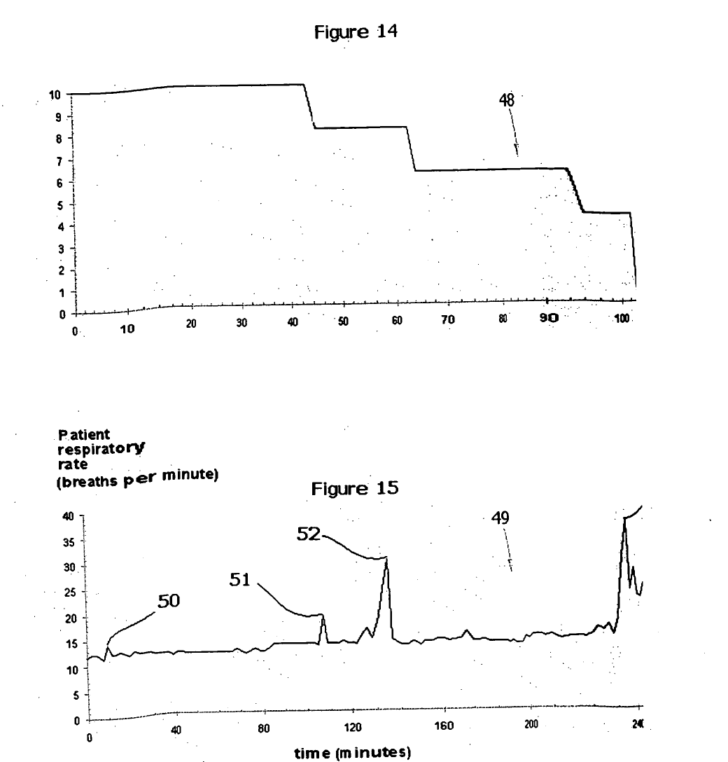

This same approach applies for analogous reasons to other bedside monitor data signals such as, for example, the mandatory respiratory rate setting. For patients using mechanical ventilation, mandatory or machine initiated breathing is also adjusted in direct proportion to the patient's ability to sustain spontaneous breaths. FIG. 12 depicts the mandatory respiratory rate versus time, with two hundred twenty six data points comprising the raw signal 46. In the case of the mandatory respiratory rate, the wavelet coefficients 47 shown in FIG. 13 are similar to the FiO₂ coefficients 44 both in quantity (e.g. fourteen non-zero coefficients 47) and character. FIG. 14 illustrates a mandatory respiratory rate value curve 48 reconstructed without loss using the fourteen wavelet coefficients 47.

Because wavelet coefficients represent details in the signal being transformed, they are a means for automatically detecting a change in the level of the raw signal data. This characteristic may be utilized in processing noisy signals where a small threshold can be used to filter out the ambient noise, leaving the larger coefficients that are typically associated with significant changes in the raw signal level.

This noise suppression feature is illustrated in FIG. 15 by respiratory data 49 obtained from a typical patient. There relatively small peaks 50 and 51, for example, representing times of high breathing rate, within the plot 49. The more prominent features are peak 52 located within the time interval of between one hundred thirty and one hundred forty minutes, and peak 53 located between two hundred thirty and two hundred forty minutes. A clinician may wish to have some notification of sudden changes such as these that occur during the course of patient monitoring. Applying a threshold to the wavelet coefficients that excludes those coefficients that are relatively small has the effect of substantially removing artifact from the signal, leaving those peaks that are significant.

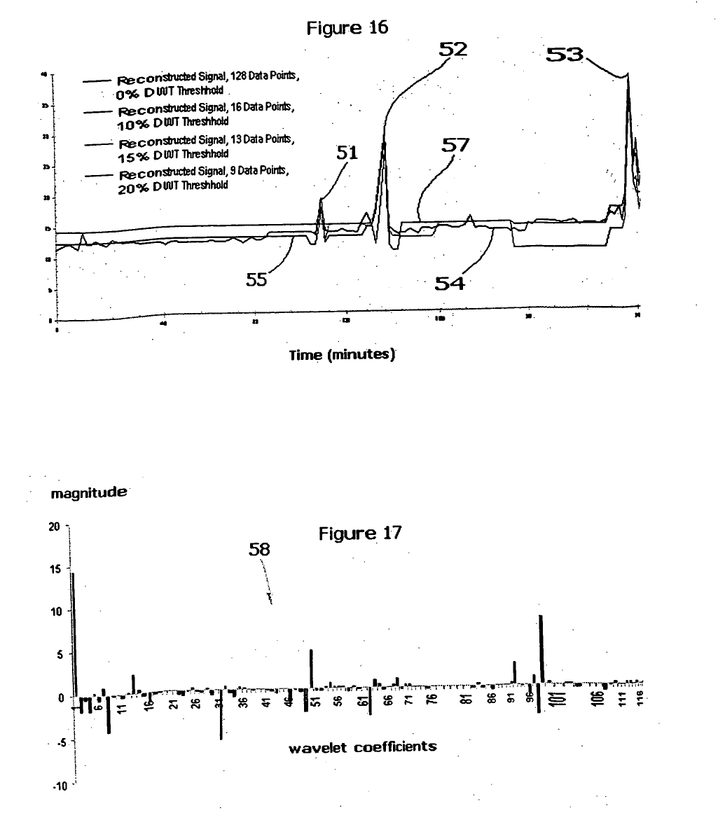

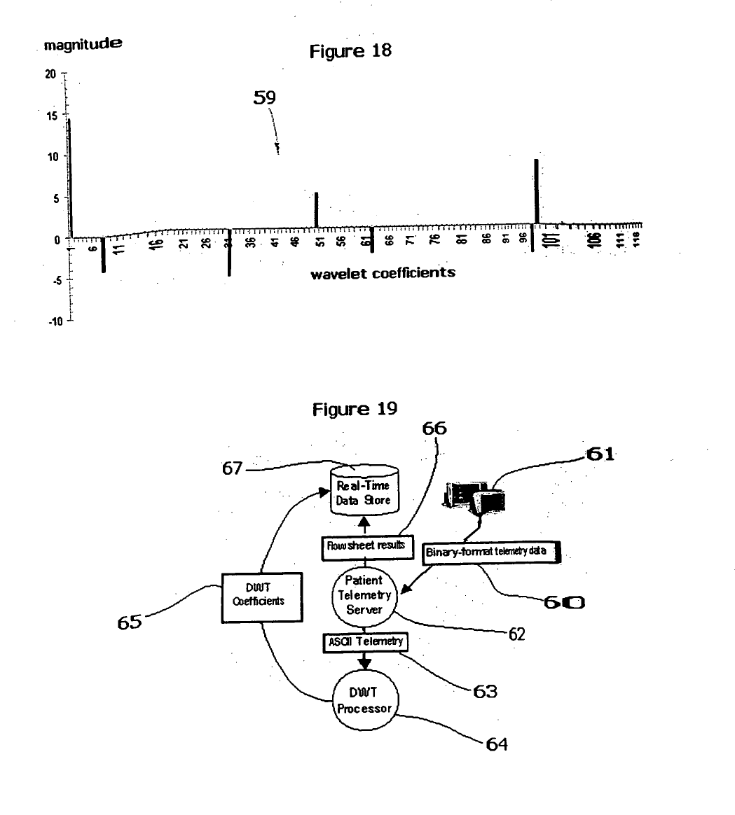

In FIG. 17, a plot 58 of the complete set of wavelet coefficients generated, i.e. with zero threshold K, is depicted. In FIG. 18, a plot 59 of the non-zero wavelet coefficients associated with a twenty percent threshold K is depicted. Four overlaid reconstructions 54, 55, 56 and 57 of the original data signal 49 are shown in FIG. 16. Curve 54 represents the signal reconstructed without loss from the complete set of wavelet coefficients, i.e. the coefficients 58 illustrated in FIG. 17. Curve 55 illustrates the signal reconstructed from wavelet coefficients filtered with a ten percent threshold K. Curve 56 illustrates the signal reconstructed from wavelet coefficients filtered with a fifteen percent threshold K. And curve 57 illustrates the signal reconstructed from the wavelet coefficients filtered with a twenty percent threshold K, i.e. the coefficients 59 illustrated in FIG. 18. Even as the threshold K is set in excess of ten percent,.the large signal peaks 51, 52 and 53 are still preserved in magnitude and location.

FIG. 19, illustrates a discrete wavelet transform processing system. Typically, real time telemetry data 60 is transmitted from the bedside monitors 61 to the local charting and database management tools operating within a patient telemetry server 62. As described above, existing systems extract samples of the telemetry data 60 at fixed time intervals, such as every fifteen minutes. These samples are supplied to the real time data storage device 67 as flow sheet or assessment sheet results 66. It is also possible for the real time data store 67 to store the complete record of the real time telemetry data 60 received from the bedside monitors 61.

The patient telemetry server 62 also produces an ASCII (American Standard Code for Information Interchange) data stream 63 which is sent directly to a communications interface of a discrete wavelet transform (DWT) processor 64. This ASCII data stream carries patient parameter data represented by a medically significant signal from a patient monitoring device attached to a patient. The DWT processor 64 is responsible for performing a transform of the patient parameter data signal to a generate a data stream consisting of a plurality of components, e.g. a DWT coefficient data stream 65, representing the patient parameter data. The component coefficient data is subsequently stored together with the real time assessment sheet data 66 in the real time data store 67.

Typically, the patient telemetry server 62, the DWT processor 64 and the real time data store 67 are implemented in a computer or processor system. As used herein, a processor operates under the control of an executable application to (a) receive information from an input information device, (b) process the information by manipulating, analyzing, modifying, converting and/or transmitting the information, and/or (c) route the information to an output information device. A processor may use, or comprise the capabilities of, a controller or microprocessor, for example. The processor may operate with a display processor or generator. A display processor or generator is a known element for generating signals representing display images or portions thereof. A processor and a display processor comprises any combination of, hardware, firmware, and/or software.

An executable application as used herein comprises code or machine readable instructions for conditioning the processor to implement predetermined functions, including those of an operating system, healthcare information system or other information processing system, for example, in response user command or input. An executable procedure is a segment of code or machine readable instruction, sub-routine, or other distinct section of code or portion of an executable application for performing one or more particular processes. These processes may include receiving input data or parameters, performing operations on received input data or performing functions in response to received input parameters, and providing resulting output parameters. A user interface comprises one or more display images, generated by the display processor under the control of the processor, enabling user interaction with a processor or other device.

The DWT processor 64 may also perform one or more mathematical operations on the raw signal data 60 in addition to the discrete wavelet transform: e.g. a Fast Fourier Transform (FFT), a Discrete Cosine Transform (DCT), signal averaging, and/or Kalman filtering. The DWT processor 64 may further filter the component (e.g. DWT coefficients) data stream based on criteria, e.g. a threshold, to reduce noise level or to identify a desired signal artifact. As described above, the criteria, e.g. threshold, may be based on a statistical significance calculation. Also as described above, this filtering may result in excluding components (e.g. DWT coefficients) from the data stream. This filtered data stream may be stored in the real time data store 67.

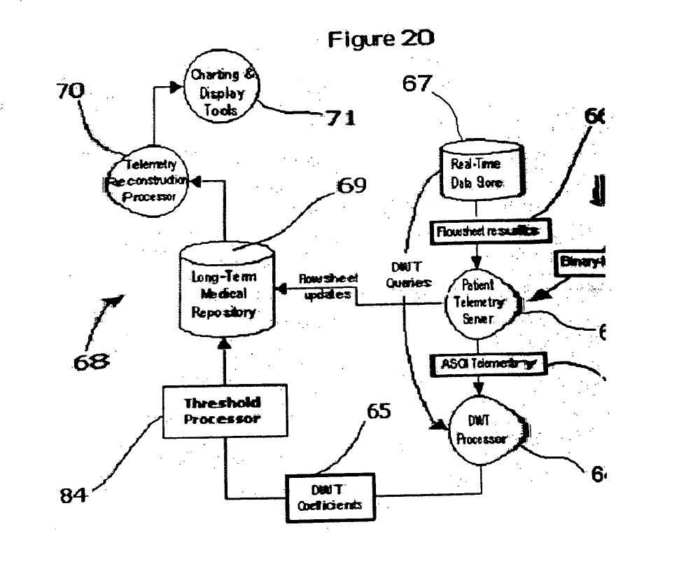

FIG. 20 is a diagram of a discrete wavelet transform processing system operating within a departmental information system 68. In FIG. 20, a user of the departmental information system 68 may request the assessment or flow sheet update data 66 from the patient telemetry server 62, or the discrete wavelet coefficients 65 from the DWT processor 64. In response to a request for the DWT coefficients 65, the DWT processor 64 accesses the real time data storage device 67 from which the coefficients 65 are retrieved, in part or in their entirety: The retrieved coefficients are sent to a telemetry reconstruction processor 70 via the threshold processor 84 and the long term medical repository 69. The threshold processor 84 allows the user to selectively filter the component stream, thus performing the filtering described in detail above. The resulting filtered DWT coefficients are stored in the long term medical repository 69.

The telemetry reconstruction processor 70 performs the inverse wavelet transform on the retrieved coefficients and permits the user to view the reassembled or approximated data signal via the charting and display tools 71, which may operate as a display processor for presenting an image of the signal for the user. Telemetry data reconstruction using the coefficients 65 is possible because the Haar matrix (or set of Haar matrices) is advantageously universal. Regardless of the particular signal data, the Haar matrix has the same form. Therefore, the Haar matrix or matrices may be permanently stored in the telemetry reconstruction processor 70 or the long term medical repository 69. In practice, one of the master files in the system 68 can be a Wavelet Transform Master (WTM) file, which is the collection of Haar matrices of various sizes used to accommodate wavelet quantities retrieved for signal reconstruction. Thus, the wavelet coefficients 65 are the data components that need to be supplied from the real time data store 67 to the enterprise user.

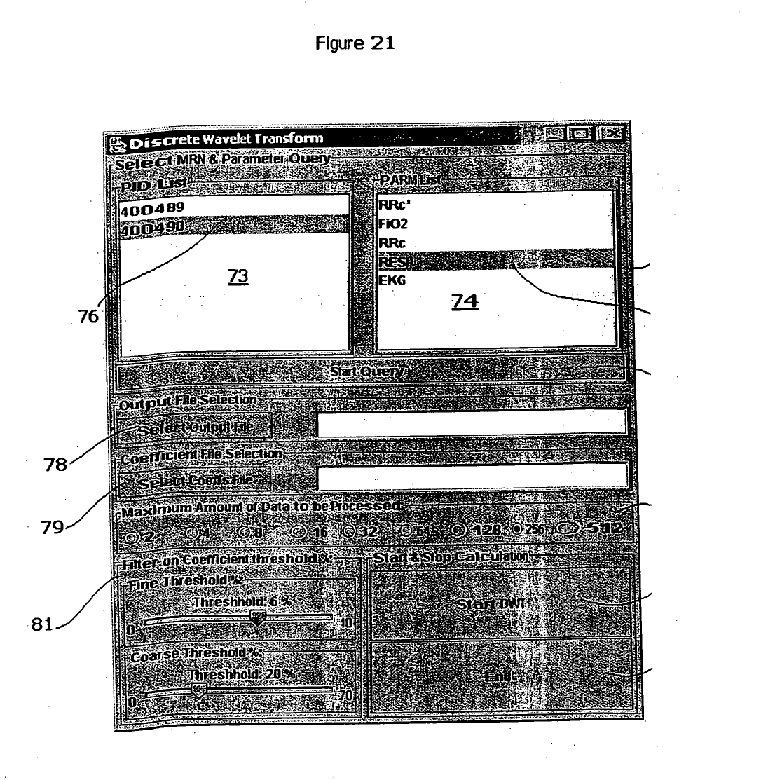

FIG. 21 illustrates a user interface 72 for accessing and controlling the operation of the discrete wavelet transform processor 64 (of FIG. 19). Data representing the image of the user interface 72 may be generated by the charting and display tools 71 (of FIG. 20), and the image of this user interface 72 may be displayed on the same display device as used to display the reconstructed patient parameter signal. A listener subprocess in the DWT processor 64 retrieves ASCII telemetry packets 63 from the patient telemetry server 62 and extracts patient demographic and vital statistics information, and the physiological parameters being monitored, for the patients. The data is written to a database in the DWT processor 64.

Operation of interface 72 begins by selecting a patient identification (PID), which may, for example, be a medical record number, for a desired patient from a list in window 73. The patient identifier (PID) list window 73 is populated automatically based upon the patient data in the ASCII packets 63 retrieved from the patient telemetry server 62. Available patient identifiers are placed in the PID list window 73. Once a patient identifier 76 is selected, the associated physiological parameters being monitored on that patient are automatically listed in the parameter (PARM) list window 74. A desired physiological parameter is selected from the list in region 74 of the interface 72. In the example shown, the PID “400490” 76 and the physiological parameter “RESP” 75 have been highlighted or selected.

Activating the Start Query button 77 begins operation of the DWT processor 64 (FIG. 19). Activating the button 77 opens a port from the patient telemetry server 62 to the DWT processor 64. The DWT processor 64 does not need to be physically located in or near the patient telemetry server 62. Instead, communication may be accomplished via TCP/IP over a standard Ethernet connection. The user is prompted to select both a signal reconstruction output file name 78 and a wavelet coefficient file name 79. The user may choose to locate these files within the current directory of the DWT processor 64 (the default directory) or elsewhere, including on a shared hard drive accessible to the enterprise information system 68. The user specifies the desired order of the DWT process within window 80, that is the number of samples of the original physiological signal to translate to wavelet coefficients at a time. One skilled in the art understands that this number of samples is to be a power of two for the Haar matrices to apply. The user may also manually specify within the threshold percentage window 81 a desired wavelet exclusion threshold K (of equation 11). Alternatively, a dedicated threshold detection processor (e.g. 84 of FIG. 20) may be employed to apply a threshold determination protocol or algorithm for automatically setting and/or maintaining a threshold value K that is appropriate to the particular raw signal being examined.

When the parameters are selected and/or specified, as described above, the Start DWT button 82 may be activated. In response, DWT processor 64 begins to calculate DWT coefficient data 65 based on the physiological parameter data samples in the ASCII packet signal 63 and to write these coefficients to the real time data storage location 67 (FIG. 19). The user may stop calculating DWT coefficient data 65 and exit the interface 72 by activating the END button 83.

The foregoing examples and descriptions are presented as illustrations of the present invention. In particular, the present invention lends itself to automation, and can be incorporated in a wide variety of data processing schemes using many different software implementations. A person of ordinary skill in the data processing field appreciates that numerous different approaches and techniques may accomplish the novel characteristics set forth herein without departing from the scope of the present invention. For example, one skilled in the art understands that all elements illustrated in FIG. 19 and FIG. 20 may be implemented in any combination of hardware, firmware and/or software.

1. A processing system for patient medical parameters, comprising:
a communication interface for acquiring patient parameter data comprising a medically significant signal from a patient monitoring device attached to a patient;
a transform processor for converting said medically significant signal into a plurality of components using a transform;
a filter for filtering said components to exclude components based on criteria to provide filtered components; and
an inverse transform processor for inverse-transforming said filtered components to provide a representation of said signal.

2. A system according to claim 1, wherein said filter excludes particular components based on criteria including a statistical significance calculation and a predetermined threshold identifying at least one of, (a) a desired signal artifact and (b) a noise level.

3. A system according to claim 1, including
a display processor for presenting said signal artifacts in an image representation to a user; and,
a threshold selection processor enabling a user to selectively exclude an artifact from said medically significant signal.

4. A system according to claim 1, including a generator for creating data representing at least one displayed user interface image supporting user selection of:
a patient;
an associated particular patient parameter type; and
an associated predetermined filtering criteria.

5. A system according to claim 1, wherein said filter excludes said components below a predetermined magnitude threshold.

6. A system according to claim 1, wherein said transform comprises at least one of: (a) a wavelet transform, (b) an FFT, (c) a DCT, (d) signal averaging, and (e) Kalman filtering.

7. A system according to claim 1, wherein said components include time and magnitude domain representative coefficients, wherein signal magnitude and temporal location are substantially preserved through the transformation process.

8. A system according to claim 2, further comprising a data store, the data store being adapted to store at least one of, (a) all parameter data generated by a patient monitoring device, (b) selected patient parameter data generated by the patient monitoring device, and (c) components used by the inverse transform processor to provide a representation of the medically significant signal.

9. A system according to claim 8, wherein the selected parameter data generated by the patient monitoring device is used to create an assessment sheet.

10. A system according to claim 9, wherein the representation of the medically significant signal created by the inverse transform processor substantially includes at least the selected parameter data present in the assessment sheet.

11. A system according to claim 10, wherein said filter excludes particular components representing temporally adjacent patient parameter data having substantially identical values.

12. A method for processing raw data streams produced by a biomedical monitoring device, wherein the raw data streams are processed by performing the following:
inputting the raw data streams to a telemetry server having processing means capable of dividing each raw data stream into at least a first component and a second component;
forwarding the first component to a storage device capable of storing substantially all data values present in the raw data stream; and
forwarding the second component to a transform processor capable of assigning a relative significance to each data value present in the raw data stream.

13. A method according to claim 12, further comprising the transform processor generating a plurality of coefficients, each coefficient characterizing a magnitude of a data value present in the raw data stream.

14. A method according to claim 13, further comprising forwarding substantially all of the coefficients to a threshold processor for setting a threshold value that excludes a set of coefficients having a relative contribution to the data values present in the raw data stream that is less than the threshold value.

15. A method according to claim 14, further comprising the threshold processor setting a threshold value that excludes a set of coefficients representing temporally adjacent substantially repeating raw data values.

16. A method according to claim 14, further comprising the threshold processor setting a threshold value that preserves a set of coefficients representing data values present in the raw data stream that indicates a significant characteristic of the data stream.

17. A method of reducing data transmission and storage requirements in a telemetry processing system used for collecting and displaying a nonstationary event, comprising:
collecting substantially all data values present in a signal that characterizes the nonstationary event;
assigning a relative contribution value to each data value present in the signal;
excluding each data value assigned a relative contribution value having a relatively small effect on the signal, thereby creating a set of excluded data values;
including each data value assigned a relative contribution value having a relatively large effect on the signal, thereby creating a set of included data values; and
constructing an approximation of the signal using the set of included data values.

18. A method according to claim 17, further comprising excluding each relative contribution value representing a data value having a magnitude that is substantially identical to a magnitude of an adjacent data value.

19. A method according to claim 17, further comprising extracting a characteristic of the nonstationary event by identifying relative contribution values associated with the characteristic.

20. A method according to claim 19, further comprising:
storing the set of included relative contribution values in a long term data storage repository; and
discarding the set of excluded relative contribution values, thereby reducing an absolute storage requirement necessary to reconstruct the approximation of the signal.
